(19) United States
(12) Patent Application Publication
(10) Pub. No.: US 2023/0298726 A1
(43) Pub. Date: Sep. 21, 2023

(54) SYSTEM AND METHOD TO PREDICT PERFORMANCE, INJURY RISK, AND RECOVERY STATUS FROM SMART CLOTHING AND OTHER WEARABLES USING MACHINE LEARNING

(71) Applicant: Vayu Technology Corp., Los Angeles, CA (US)

(72) Inventors: Karan Kanchan, Glendale, CA (US); Ganesh Prasad, San Jose, CA (US); Ami Mehta, Glendale, CA (US); Harsh Holalad, Bangalore (IN); Prathamesh Mantri, Kalyan (IN)

(21) Appl. No.: 18/124,565

(22) Filed: Mar. 21, 2023

Related U.S. Application Data
(60) Provisional application No. 63/321,778, filed on Mar. 21, 2022.

(51) Int. Cl. G16H 20/30; G06N 20/00; G16H 40/67; G16H 50/30; G16H 70/20

(52) U.S. Cl. CPC G16H 20/30; G06N 20/00; G16H 40/67; G16H 50/30; G16H 70/20

(57) ABSTRACT

A portable, wearable multi-component measurement and analysis system and method can be utilized for collecting and analyzing bio-mechanical and human gait analysis data while performing any physical activity. The system and method are used to enhance biomechanics databases, and use machine learning to develop predictive models, while further correlating that to data collected by consumer devices and smart apparel, to ultimately use data from consumer devices only, to provide insights (measurements and diagnostics) and feedback (recommendations) for optimizing performance, preventing injuries and expediting recovery to device owners and others (persons with whom device owner would like to share information).

### PRIORITY CLAIM

This application claims priority to and the benefit of U.S. Provisional Patent Application No. 63/321,775, titled System and Method to Predict Performance, Injury Risk, and Recovery Status from Smart Clothing and Other Wearables Using Machine Learning, and filed on Mar. 21, 2022, at the United States Patent and Trademark Office, the entire content of which is incorporated by reference herein as if fully set forth below in its entirety for all applicable purposes.

### TECHNICAL FIELD

Various examples of the present disclosure relate to the field of bio-mechanics and/or human gait analysis.

### BACKGROUND

Applicant is the owner of U.S. Pat. No. 10,194,837 (the '837 patent) the entirety of which is incorporated by reference herein. The '837 patent is directed to a portable, wearable, and multicomponent device, constructed with one or more sensors, creating a database of biomechanics and gait information.

As discussed in the '837 patent, gait analysis is typically conducted in a lab setting with high-speed cameras and force plates. Luminescent markers may be used, which may be bulky, complex, and expensive. The existing configuration has mobility challenges and provides limited, time-based data for any activity. The current mechanism that tracks human activity, mobility, and posture over a defined period (e.g., 15 to 20 minutes in a lab) is not sufficient to provide a thorough and robust source of data for analysis. The mechanical devices used to gather the necessary data to make analytical assessments may contribute to the deficiencies of the current methods.

Consumer devices today (phones, watches, etc.) can provide insights limited to steps, calories, heart rate, and sleep but cannot provide insights into musculoskeletal/biomechanics/gait insights, which are critical in understanding and developing solutions to optimize performance, prevent injuries and expedite recovery. Current technology solutions that can provide deeper musculoskeletal/biomechanics/gait insights are often powered by either one or a combination of external equipment like sensor force plates, cameras, etc. thereby limiting the ability to service a larger audience at anytime and anywhere.

What is needed is a system and method that enables generating deeper musculoskeletal/biomechanics/gait insights using data from consumer devices (phones, watches, etc.) and smart apparel (a nascent and upcoming market).

### BRIEF SUMMARY

The following presents a simplified summary of one or more aspects of the present disclosure to provide a basic understanding of such aspects. This summary is not an extensive overview of all contemplated features of the disclosure. It is intended neither to identify key or critical elements of all aspects of the disclosure nor to delineate the scope of any or all aspects of the disclosure. Its sole purpose is to present some concepts of one or more aspects of the disclosure in a simplified form as a prelude to the more detailed description presented below.

In one example, a system for collecting data related to user movements is provided. The system includes at least one wearable device, wherein the at least one wearable device comprises at least one sensor to collect low-fidelity data; and one or more servers for receiving the low-fidelity data via a communication network. The one or more servers comprises one or more databases for storing instructions for processing the low-fidelity data; one or more processors, in communication with the one or more databases, configured to execute the instructions to perform a method, including: collect the low-fidelity data from the at least one wearable device; transmit the low-fidelity data from the at least one wearable device to the one or more servers; extrapolate the low-fidelity data using predictive analysis developed by machine learning models trained with high-fidelity data; amplify the low-fidelity data; provide personalized recommendations to a user using the amplified low-fidelity data.

According to one aspect, the at least one wearable device includes a consumer device.

According to another aspect, the low-fidelity data is transmitted securely and anonymously to the at least one server.

According to yet another aspect, the personalized recommendations include performance, injury risk, and recovery status of the user.

According to yet another aspect, the at least one wearable device includes a wireless transmitter configured to transmit the low-fidelity data collected.

According to yet another aspect, the at least one wearable device includes a consumer device and an article of clothing, an accessory or an implant.

According to yet another aspect, the at least one sensor includes at least one of an accelerometer, a gyroscope, a magnetometer, and a heart rate sensor.

According to yet another aspect, the one or more processors is further configured to: measure cognitive health and balance of the user; assess the cognitive health and balance of the user; and quantity the cognitive health and balance of the user.

According to yet another aspect, the system further comprises an artificial chat bot in communication with the one or more servers to assess all performance, injury risk, and recovery insights to answer questions from the user in real-time.

According to yet another aspect, multiple trials, tests, assessments, and reports are utilized to answer the questions in real-time.

According to another example, a system for collecting data related to user movements is provided. The system comprises at least one wearable device, wherein the at least one wearable device comprises at least one sensor to collect low-fidelity data; at least one consumer device, wherein the at least one consumer device comprises at least one sensor to collect low-fidelity data; one or more servers for receiving the low-fidelity data via a communication network, the one or more servers comprising: one or more databases for storing instructions for processing the low-fidelity data; one or more processors, in communication with the one or more databases, configured to execute the instructions to perform a method, including: collect the low-fidelity data from the at least one wearable device; transmit the low-fidelity data from the at least one wearable device to the one or more servers; extrapolate the low-fidelity data using predictive analysis developed by machine learning models trained with high-fidelity data; amplify the low-fidelity data; provide personalized recommendations to a user using the amplified low-fidelity data.

According to one aspect, the low-fidelity data collected from the at least one wearable device and the at least one consumer device is transmitted securely and anonymously to the at least one server.

According to another aspect, the at least one wearable device and the at least one consumer device communicate with each other directly.

According to yet another aspect, the at least one wearable device includes an article of clothing, an accessory, or an implant.

According to yet another aspect, the at least one wearable device includes a wireless transmitter configured to transmit the low-fidelity data collected.

According to yet another aspect, the low-fidelity data is transmitted securely and anonymously to the at least one server.

According to yet another aspect, the one or more processors is further configured to: measure cognitive health and balance of the user; assess the cognitive health and balance of the user; and quantity the cognitive health and balance of the user.

### BRIEF DESCRIPTION OF THE DRAWINGS

The features, nature, and advantages of the present aspects may become more apparent from the detailed description set forth below when taken in conjunction with the drawings in which like reference characters identify correspondingly throughout.

FIG. 1 is a flow diagram showing the collection of limited biomechanics data (“low-fidelity data”) from wearable and/or consumer devices.

FIG. 2 is a flow diagram showing high quality analysis and predictions of performance improvement, injury risk and recovery which require a lot of data, and high-quality data (“high-fidelity data”).

FIG. 3 is a flow diagram illustrating a method for accessing sparse or low-fidelity data from one or more consumer devices (phone, watches, etc.) and/or smart apparel and the training of machine learning models with high fidelity data, which is then used to amplify low fidelity data, thereby supercharging consumer devices to provide sophisticated biomechanics and musculoskeletal insights (which go beyond their default capabilities).

FIG. 4 is a flow diagram illustrating a method of predicting performance, injury risk, and recovery status from smart clothing and other wearables using machine learning and how machine learning models are trained using private corpus data and other public corpus of data (from open-source data/publications available) to amplify low fidelity data from consumer devices to provide sophisticated and actionable biomechanics insights.

FIG. 5 is a schematic of a wearable device according to an example of the present disclosure.

FIG. 6 is a schematic of server according to an example of the present disclosure.

FIG. 7 is a schematic of a consumer device according to an example of the present disclosure.

FIG. 8 is a schematic illustration of a system according to an exemplary embodiment of the present disclosure.

FIG. 9 is a flow chart illustrating an example of a process of providing sophisticated analysis and insights on various aspects of the body leading to more relevant and useful predictions on performance improvement and injury risk while continuously monitoring the progress, according to some aspects of the disclosure.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts. As used herein, a reference to an element in the singular contemplates the reference to the element in the plural.

### Terms

As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. The term “exemplary” is used in the sense of “example,” rather than “ideal.”

The terms “A”, “an,” and “the” and similar referents used herein are to be construed to cover both the singular and the plural unless their usage in context indicates otherwise. The word “exemplary” is used herein to mean “serving as an example, instance, or illustration.” Any implementation or embodiment described herein as “exemplary” is not necessarily to be construed as preferred or advantageous over other embodiments or implementations. Likewise, “embodiments” does not require that all embodiments include the discussed feature, advantage, or mode of operation.

The term “aspect” does not require that all aspects of this disclosure include the discussed feature, advantage, or mode of operation.

The term “coupled” is used herein to means the direct or indirect coupling between two objects. For example, if object A physically touches or couples to object B, and object B touches or couples to object C, then objects A and C may still be considered coupled to one another, even if they do not directly physically touch each other.

The terms “wearable” and “wearable devices” may refer to any type or form of computing device that is worn by a user as part of an article of clothing, an accessory, and/or an implant. Examples of wearable devices include, without limitation, headsets, headbands, head-mounted displays, glasses, frames, variations, smart watches, or combinations of one or more of the same, and/or any other suitable wearable devices.

The term “consumer device” may refer to a device capable of wireless telecommunication and may be, but is not limited to, a mobile phone, smart watch, smart glasses, rings, bands, or any other consumer device to the extent that it is capable of wireless telecommunication. “Consumer devices” may be classified as wearables/wearable devices when carried on the body, for example, in a pants or shirt pocket.

Low-fidelity data (LoFi) includes any data that was produced by a person or Stochastic Process that deviates from the real-world system of interest.

High-fidelity data (HiFi) includes data that was produced by a person or Stochastic Process that closely matches the operational context of interest.

The terms “computer-readable medium” as used herein refers to any tangible storage that participates in providing instructions to a processor for execution. Such a medium may take many forms, including but not limited to, non-volatile media, volatile media, and transmission media. Non-volatile media includes, for example, NVRAM, or magnetic or optical disks. Volatile media includes dynamic memory, such as main memory. Common forms of computer-readable media include, for example, a floppy disk, a flexible disk, hard disk, magnetic tape, or any other magnetic medium, magneto-optical medium, a CD-ROM, any other optical medium, punch cards, paper tape, any other physical medium with patterns of holes, a RAM, a PROM, and EPROM, a FLASH-EPROM, a solid state medium like a memory card, any other memory chip or cartridge, or any other medium from which a computer can read. When the computer-readable media is configured as a database, it is to be understood that the database may be any type of database, such as relational, hierarchical, object-oriented, and/or the like. Accordingly, the disclosure is considered to include a tangible storage medium and prior art-recognized equivalents and successor media, in which the software implementations of the present disclosure are stored.

The terms “central processing unit”, “processor”, “processor circuit”, and “processing circuit”, and variations thereof, as used herein, are used interchangeably and include, but are not limited to, a general purpose processor, a digital signal processor (DSP), an application-specific integrated circuit (ASIC), a field programmable gate array (FPGA) or other programmable logic component, discrete gate or transistor logic, discrete hardware components, or any combination thereof designed to perform the functions described herein. A general-purpose processor may include a microprocessor, as well as any conventional processor, controller, microcontroller, or state machine. The processor may also be implemented as a combination of computing components, such as a combination of a DSP and a microprocessor, a number of microprocessors, one or more microprocessors in conjunction with a DSP core, an ASIC and a microprocessor, or any other number of varying configurations. These examples of the processors are for illustration and other suitable configurations within the scope of the disclosure are also contemplated. Furthermore, the processor may be implemented as one or more processors, one or more controllers, and/or other structure configured to execute executable programming.

The terms “determine,” “calculate,” and “compute,” and variations thereof, as used herein, are used interchangeably, and include any type of methodology, process, mathematical operation, or technique.

The term “module” as used herein refers to any known or later developed hardware, software, firmware, artificial intelligence, fuzzy logic, or combination of hardware and software that is capable of performing the functionality associated with that element.

This disclosure provides sophisticated engineering to positively contribute to one or more of the fields of bio-mechanics, gait analysis, and any other domain that requires measurement and/or analysis of human body motion. Devices and methods of the present disclosure may allow users to avoid travel time to an analysis lab, expand consultation with doctors beyond geographic constraints, provide the freedom to measure and/or analyze a variety of physical activities over a longer period of time, and provide the freedom to conduct measurements and/or analysis at convenient times and locations for the user, such as in the natural environment of the analyzed activity and/or daily routine. Each of the embodiments described herein may include one or more of the features described in connection with any of the embodiments described herein.

### Overview

The present disclosure is directed to a system and method to enhance the database as disclosed in the '837 patent along with other biomechanics databases (created by similar or alternate methods/devices/technologies), and use machine learning to develop predictive models, while further correlating that to data collected by consumer devices and smart apparel, to ultimately use data from consumer devices only, to provide insights (measurements and diagnostics) and feedback (recommendations) for optimizing performance, preventing injuries and expediting recovery to device owners and others (persons with whom device owner would like to share information).

Examples of the present disclosure include systems and methods that use single and/or multi-component wearable devices that work together, interdependently, or independently to collect, analyze, and/or give feedback on a user's bio-mechanical activity while the user is performing any particular exercise.

Healthcare is very expensive today and remains inaccessible (driven by loads of processes imposed by payers (insurance companies) and providers (hospitals)). The system and methods of the present disclosure is that it redefines healthcare via digital health by enabling/democratizing lab quality and medical grade assessments by using existing and common devices owned by people at home, thereby making healthcare affordable and highly accessible.

Furthermore, in addition to helping identify problems objectively, system and methods of the present disclosure are also capable of making recommendations on ‘what to do to fix the problem”, which can be for example but not limited to exercise recommendations, rehabilitation and therapy protocols, sleep guidance, workouts, nutrition plans, mental health coaching, etc., wherein the user shall be continuously prompted and encouraged to complete the tasks with an interactive and/or gamified experience, wherein a user is taken through a guided learning and rewarding process throughout the day/week/month/year to improve overall performance, injury risk, and/or recovery status.

Additionally, the system and methods of the present disclosure assist users in tracking progress for every performance, injury risk, and recovery insight generated to see what is getting better and/or deteriorating over time.

### Low-Fidelity Data Collection

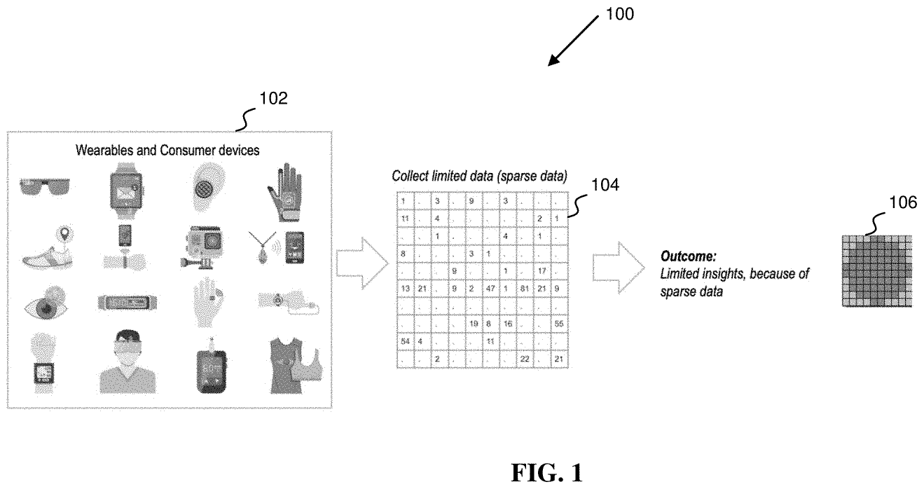

FIG. 1 is a flow diagram 100 showing the collection of limited biomechanics data (“low-fidelity data”) from wearable and/or consumer devices. As shown, wearable and/or consumer devices 102 can be used to collect limited data (low-fidelity data) 104 related to user movement, such as steps, calories, heart rate, and sleep. While this data can be useful and provide limited insights 106, it cannot provide insights into musculoskeletal/biomechanics/gait insights, which are critical in understanding and developing solutions to optimize performance, prevent injuries and expedite recovery.

### High-Fidelity Data Collection

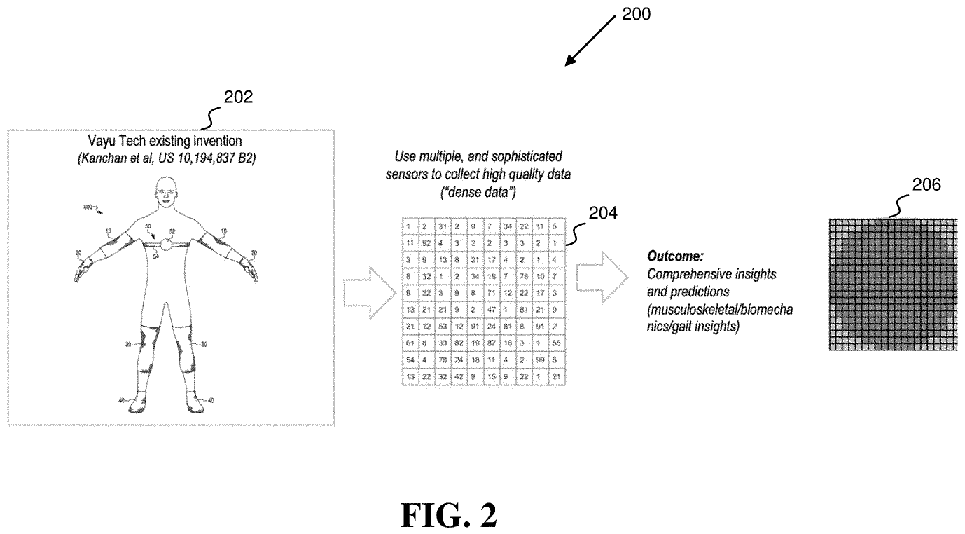

FIG. 2 is a flow diagram 200 showing high quality analysis and predictions of performance improvement, injury risk and recovery which require a lot of data, and high-quality data (“high-fidelity data”). The analysis and predications are discussed in the '837 patent. However, collection of “high-fidelity data” requires sophisticated sensors and is not commercially viable for mass market implementation. The present disclosure provides a system and method of generating “high fidelity data” from existing “low-fidelity data”. More specifically, the present invention marries the existing high-fidelity data that is collected by products disclosed in the '837 patent and other biomechanics sources (for example but not limited to data from force plates, motion capture labs, electromyography (EMG) technology, etc.) and “amplifies” the low-fidelity data, resulting in enabling consumer devices and wearables to offer sophisticated analysis and predictions using a software only approach, without the need to employ expensive sensors.

As shown, high-fidelity data may be collected 202 using devices having one or more sensors that are placed on the various localities of human body to collect bio-mechanical data, such as products disclosed in the '837 patent. The use of multiple and sophisticated sensors allows for the collection of high-fidelity data (“dense data”) 204. The high-fidelity data collected provides comprehensive insights and predictions (musculoskeletal/biomechanics/gait insights) 206.

### Accessing Low-Fidelity Data

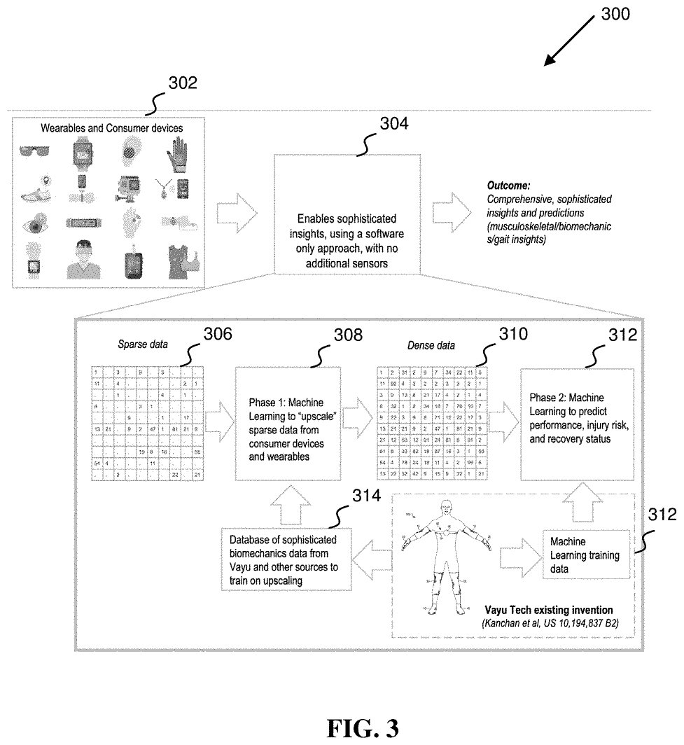

FIG. 3 is a flow diagram 300 illustrating a method for accessing sparse or low-fidelity data from one or more consumer devices (phone, watches, etc.) and/or smart apparel and the training of machine learning models with high fidelity data, which is then used to amplify low fidelity data, thereby supercharging consumer devices to provide sophisticated biomechanics and musculoskeletal insights (which go beyond their default capabilities). As shown, wearable and/or consumer devices 302 can be used to collect limited biomechanics data (low-fidelity data) related to user movement, such as GPS coordinates, step count, VO2 max, SPO2 (oxygen saturation), calories, heart rate, sleep, and any other available health, wellness, and fitness information that can be obtained from wearables/wearable devices/consumer devices. Using only the low-fidelity data collected data and no additional sensors, sophisticated insights from the data may be determined 304.

After the sparse or low-fidelity data is collected 306, this data may then be securely and anonymously transmitted from the consumer devices to one or more servers via the Internet. Next, the data is extrapolated on the one or more servers using predictive analysis developed by machine learning models trained with high-fidelity data to provide various insights, for example, movement analysis, performance insights, injury risk markers, etc. That is, using the existing sensors in consumer devices and wearables, without the need for additional sensors, machine learning can be used to teach the consumer devices and wearables to “upscale” existing data 308 into dense data 310. The dense data may be obtained using predictive analytics developed by machine learning models (trained with “high-fidelity data”) can predict performance, injury risk, and recovery status of the user 312. According to some aspect, endurance tests may be utilized to continuously monitor users over a longer duration of time for example, including but not limited to, a long walk, a marathon, a swim, and a bike ride. Additionally, according to some embodiment, users can “tag” their trials/tests/assessments/reports performed (i.e., ability to add additional context wither manually or retrieved from other sources automatically) with for example, the type of shoe worn, orthotics, prosthetics, assistive devices used while performing, the terrain on which it was performed, sleep, hydration, workload levels under which it was performed, treatment received before and after performing, nutritional factors such as food consumed before and after performing, and any other custom tag/context. This allows users to determine how specific factors affect performance and rehabilitation.

The predictive analytics or analysis developed by machine learning models as disclosed in the '837 patent 312 as well as the database of biomechanics and gait information from the '837 patent as well as other sources to train on upscaling data 314, is utilized to upscale the data.

This predictive analysis, developed by machine learning models (trained with “high-fidelity data”), can:
1. Use data from, for example but not limited to, accelerometers, gyroscopes, magnetometers, and/or heart rate sensors, using predictive analytics to measure forces acting on the body as whole and/or on different limbs (arms, legs, etc.) and/or on specific limb segments/joints, and further quantifying the ability to develop forces (a measure of strength, power, etc.) and absorb shock (a precursor to understanding injury risk) with respect to time;
2. Measure 3D movements for both segments in sagittal, frontal and transverse planes along with joints in all degrees of freedom, thereby predicting range of motion changes with respect to time (insights used to quantify mobility, alignment, flexibility, control, etc.);
3. Analyze gait (for example but not limited to ground contact time, airtime, gait cycles, gait imbalance, cadence, acceleration, breaking, velocity, etc.) and further using those insights to identify/validate whether the person(s) are wearing the appropriate footwear and if not to make suggestions for the same to improve performance, prevent injuries and/or expedite recovery;
4. Use the above combination of insights to quantify overall body's strength, power, mobility, flexibility, alignment, control, etc. and further develop learning models that can define a population specific scoring system (with respect to but not limited to age, gender, physical capabilities, athletic potential, injury history, height, weight, shoe size, etc.) to baseline, progression track and continuously monitor performance, injuries and recovery status further providing a rating/indication similar but not limited to problem, concern and/or good. This capability allows a apples-to-apples comparison between a specific user to his own demographic (aka a youth tennis player dealing with a shoulder injury is best compared to and cross referenced with shoulder injury causes/risks/recovery status in a larger group of youth tennis players and NOT for example a geriatric population that doesn't play sport at all);
5. Use audio, visual, haptic and biofeedback via a device speaker, headphone connection, touchscreen, vibrations, etc. to notify and/or provide feedback to persons on but not limited to their performance, injuries, and recovery status along with recommendations/suggestions to address them;
6. Enable device owners to integrate insights into device applications aggregating health and other information and further share that data/insights/reports (with one specific insight or hybrid insights correlating multiple insights) with other persons (anyone but not limited to coaches, trainers, doctors, therapists, family members, etc.) in real-time and/or post facto;
7. Use insights (biomechanics, gait, etc.) generated along with other data available (for example but not limited to heart rate, sleep, etc.) to measure/assess/quantify cognitive health and balance (for example but not limited to quantifying and predicting concussions, fall risk, Alzheimer's, Parkinson's, Huntington's, etc. and associated risks)
8. Provide personalized recommendations, solutions and/or programs encompassing equipment/product purchases, exercises and/or therapy which will be individualized and tailored based on insights generated (for example but not limited to biomechanics, gait, etc. and related musculoskeletal or neuromuscular predictions)
9. Allow creation of Bio-NFT's (biological non-fungible tokens), which are unique digital assets created by using one's own personalized information (performance, injury risk and recovery data and insights) and permanently recorded on a blockchain or digital ledger;
10. Further allow Bio-NFT's to be exported to platforms (for example but not limited to social media, video gaming, virtual reality, augmented reality, metaverses, etc.); and
11. Further allow Bio-NFT's to be monetized on platforms that allow/enable sharing, selling, trading, contributing, etc. of digital assets.

All of the above features/capabilities apply to humans (as an independent body), humans and animals (as independent or conjoined bodies) and animals (as an independent body, with human intervention enabling animal access to consumer devices (phones, watches, etc.) and/or smart apparel).

The present disclosure may also include the creation of “protocols”, or “plans” or “packs”. These protocols can be created by a user to include a prescribed set of tests to be done, in a particular order, with a configurable number of iterations. These protocols can then be used to provide a testing experience to establish “baselines” for testing groups of people or cohorts to establish patterns of performance and injury risk.

According to one example, a “back to work” or “back to sport” or “back to play” protocol may be created that would include a series of tests or activities with “pass” and “fail” being mentioned so that when a candidate recovering from a work or sport-related injury has to be cleared to be fit, the protocol would be used to quickly and objectively ascertain readiness. Since these protocols can be applied to existing wearables and smart devices, the candidate could execute the protocols at their own convenience, say, in their homes, without having to travel to a testing facility. This method may choose to prompt individuals to perform a series of tests periodically, further choose to provide audio/video guidance to perform such tests and/or use a combination of activities/movements performed during the day to ascertain readiness.

According to another example, a “concussion detection protocol” may be created that can be used on the playing field itself, to get an objective assessment of probability of concussion. The pass/fail thresholds can be defined conservatively, so that in the case of abundant caution, the player could be removed from the field for further observation, while for all other cases, a “pass” result would enable the player to continue playing.

According to yet another example, a sophisticated “rehab” protocol may be created. This protocol would enable a clinician and a physical therapist (PT) to send the patient home and repeat the rehab protocol at their home, using off-the-shelf consumer devices and wearables, yet delivering sophisticated results. The clinician and/or PT could be alerted in case of abnormal results allowing the clinician and/or PT to intervene, thus enabling truly “remote-rehabilitation”.

According to yet another example, a “balance test” protocol may be created that would be useful for the geriatric population, for example, that could be used to assess the risk of fall. This protocol may be a single or a series of tests to be performed by the elderly upon a doctor's request, upon a caretaker's request and/or upon an automatic prompt from the device itself.

According to yet another example, a “posture detection protocol” may be created that could be used to assess posture and may further provide data/insights/feedback on how that impacts performance, injury risk and recovery along with further instructions/suggestions/recommendations to address the same.

According to yet another example, the present disclosure may also completely eliminate the need to perform a series of tests (whether guided or otherwise) by taking into consideration daily activities and movements performed by the user in order to arrive at the same conclusions as if a specific test protocol were performed.

According to yet another example, the present disclosure may use insights generated to substantiate performance, injury and recovery and further correlate that to mental and emotional well-being of persons, which may lead to suggestions such as advising persons to see experts in, for example but not limited to, physiology, psychology, and neurology.

According to yet another example, the present disclosure may allow third party health and wellness applications to leverage a single insight or a plurality of insights generated, via data sharing agreements, thereby using this method to improve their services and offerings, which may or may not be focused on musculoskeletal performance, injuries and/or recovery.

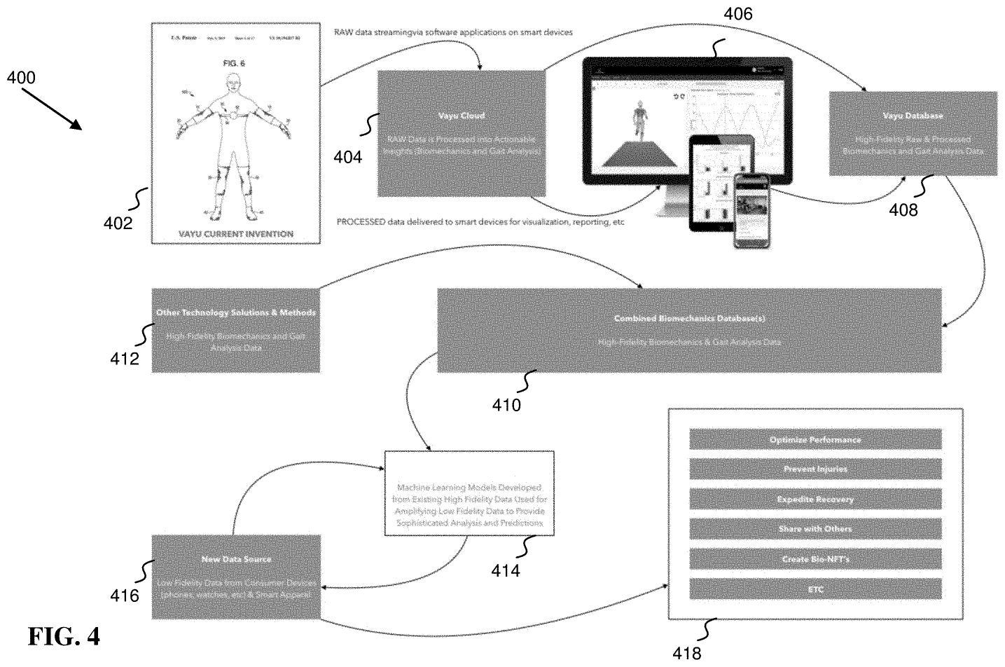

Turning to FIG. 4, a flow diagram 400 a method of predicting performance, injury risk, and recovery status from smart clothing and other wearables using machine learning and how machine learning models are trained using private corpus data (as disclosed in the '837 patent) and other public corpus of data (from open-source data/publications available) to amplify low fidelity data from consumer devices to provide sophisticated and actionable biomechanics insights is illustrated. As shown, sensors may be located on the body or in compression cloths 402. The sensors collect raw biomechanics and gait data and send the collected data to the cloud server via a smart device for processing 404. The processed data provides measurements, diagnostics, predictions, and recommendations to optimize performance, prevent injuries and expedite recovery 406. All RAW and processed data is stored in a database 408. That is, high-fidelity RAW and processed biomechanics and gait analysis data.

The RAW and processed data in the database may be accessed 410. Additionally, RAW and processed data in other biomechanics databases may be accessed 412. The data from all databases may be used to develop predictive analytics using machine learning 414. This data may then be correlated to data collected from consumer devices and smart apparel 416. Following which, data from consumer devices and smart apparel only is utilized. The data may be used to provide measurements, diagnostics, predictions, and recommendations to optimize performance, prevent injuries and expedite recovery for device owners further allowing devices owners to share data with others 418.

### Wearable Devices

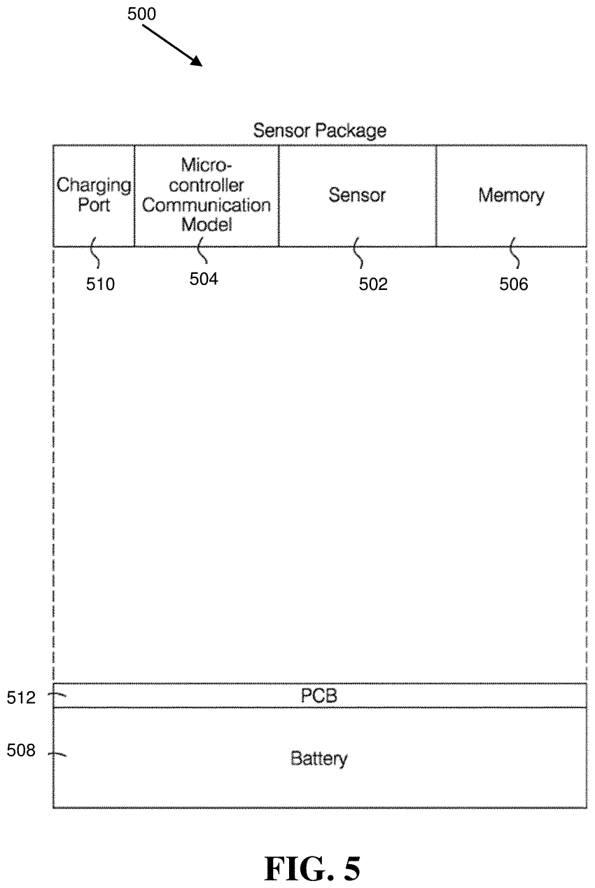

Each wearable device described herein may include one or more sensors. The sensors may be included within a sensor package having one or more sensors along with other components, or the sensors may be individual pressure sensors or other types of sensors. An exemplary sensor package in a wearable device 500 is shown in FIG. 5. The sensor packages described in connection with the wearable devices herein may the same as or may include different components than sensor package 500. The sensor package 500 may include one or more sensors 500. The sensor 500 may be a force and/or IMU (inertial measurement unit) sensor. The sensor package 500 may further include a processor 504 (e.g., an ARM-based microcontroller), a wireless or wired transmitter (e.g., a low energy module which may support Bluetooth Low Energy (BLE) or Wi-Fi) (included within processor 504), a memory chip 506, a battery 508, a charging port 510, and a primary circuit board 512. Sensors may be mounted on primary circuit board 512, which may be flexible and may conform to the curvature of the body. In some embodiments, one or more of the memory chip, wireless transmitter, and charging port may be omitted. For example, charging port may be replaced with a replaceable battery, or a wireless charging module. The sensor package may be encased in a waterproof casing/covering. In one example, one or more sensors 502 within sensor package 500 may include one or more of an accelerometer, a gyroscope, and a magnetometer. These components may facilitate calculation of a body's center of mass.

### Server

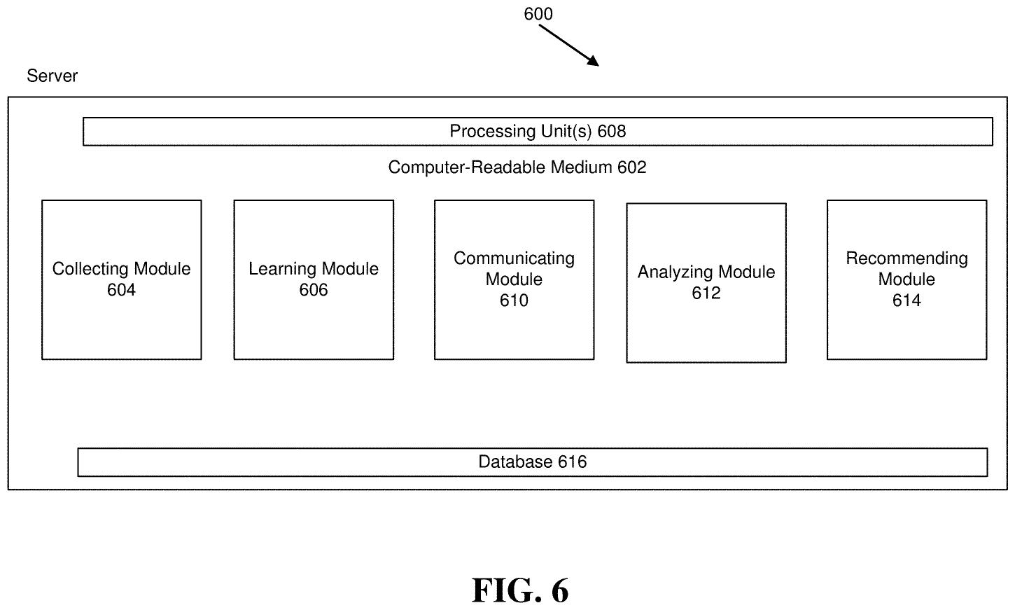

FIG. 6 is a schematic of a server according to an example of the present disclosure. Servers may include any type of computing device having one or more processing unit(s) operably connected to computer-readable media 602 such as via a bus, which in some instances can include one or more of a system bus, a data bus, an address bus, a PCI bus, a Mini-PCI bus, and any variety of local, peripheral, and/or independent buses. The computer-readable medium 602 may be a non-transitory computer-readable medium and can include one or more modules and data structures including, for example, collecting module 604, learning module 606, and other modules, programs, or applications that are loadable and executable by processing units(s) 608. In at least one example, the server 600 can additionally or alternatively include a communicating module 610, analyzing module 612, and/or recommending module 614 for performing at least some of the functions described above. The server 600 may further include a database 616 for creating a database of biomechanics and gait information. The one or more modules and data structures can be in the form of stand-alone applications, productivity applications, an OS component or any other application or software module having features that facilitate interactions between the server, consumer devices, and/or wearable devices.

### Consumer Devices

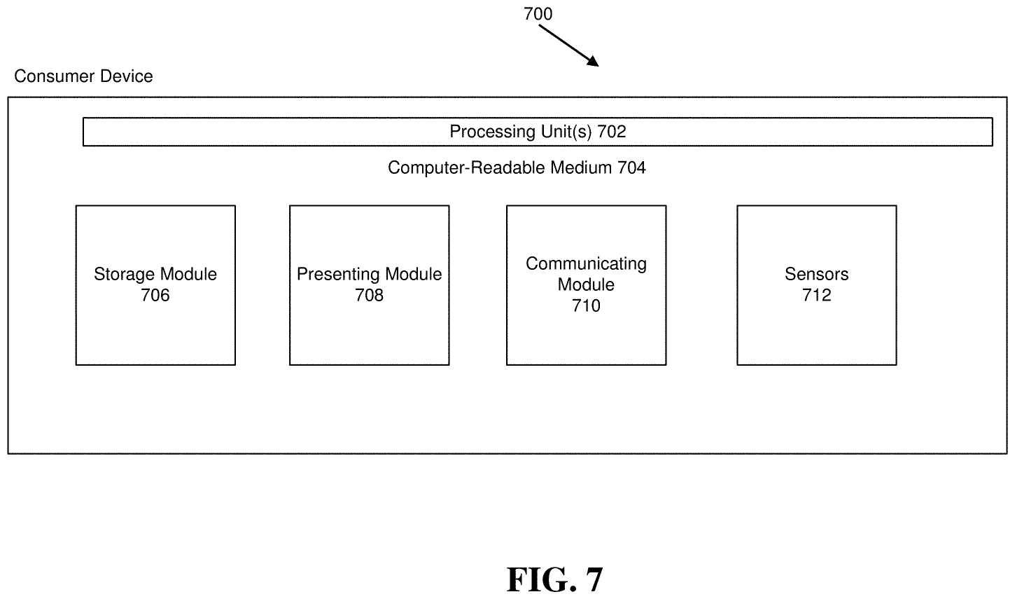

FIG. 7 is a schematic of a consumer device according to an example of the present disclosure. As discussed previously, consumer devices (phones, watches, etc.) can provide insights into steps, calories, heart rate, and sleep for example.

Consumer device(s) 700 can represent a diverse variety of device types and are not limited to any particular type of device. Consumer device(s) 700 can include any type of computing device having one or more processing unit(s) 702 operably connected to computer-readable media 704 such as via a bus, which in some instances can include one or more of a system bus, a data bus, an address bus, a PCI bus, a Mini-PCI bus, and any variety of local, peripheral, and/or independent buses. The computer-readable medium 704 may be a non-transitory computer-readable medium and can include can include one or more modules and data structures including, for example, a storage module 706, a presenting module 708, a communication module 710, one or more sensors 712, and other modules, programs, or applications that are loadable and executable by processing units(s) 702. The one or more modules and data structures can be in the form of stand-alone applications, productivity applications, an OS component or any other application or software module having features that facilitate interactions between the server, consumer devices, and/or wearable devices.

Collecting Data Using Wearable Devices and/or Consumer Devices

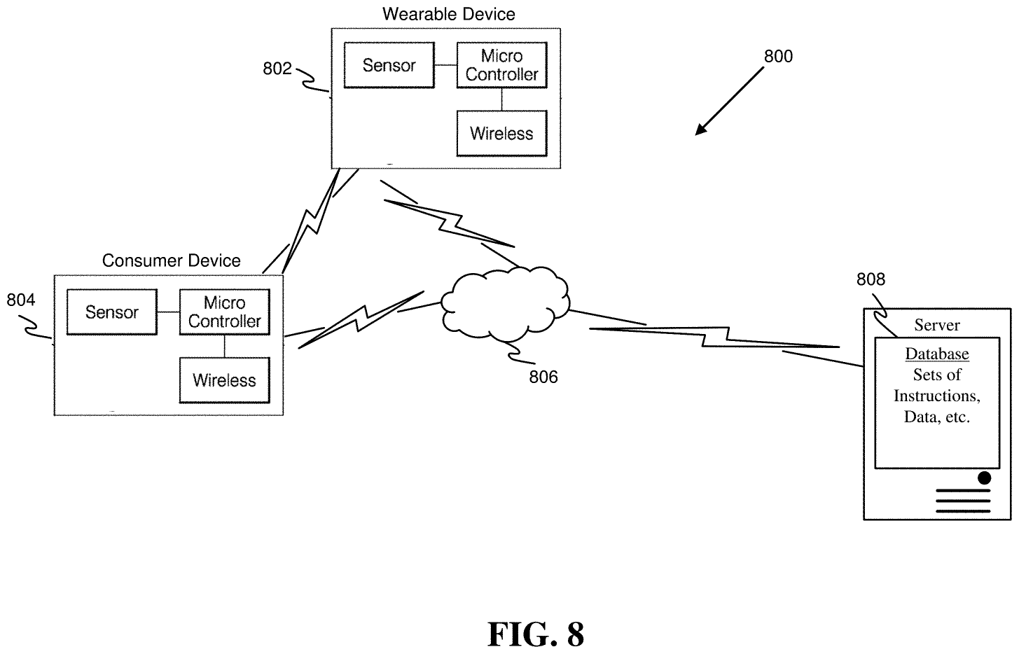

FIG. 8 is a schematic illustration of a system 800 according to an exemplary embodiment of the present disclosure. Data can be collected from a user via one or more wearable devices 802 and/or one or more consumer devices 804. As discussed previously, consumer devices 804 may be classified as wearables/wearable devices when carried on the body, for example, in a pants or shirt pocket.

Each wearable device 802 described herein may include one or more sensors. The sensors may be included within a sensor package having one or more sensors along with other components, or the sensors may be individual pressure sensors or other types of sensors. An exemplary sensor package 500 is shown in FIG. 5. Furthermore, each consumer device 804 described herein may include one or more sensors. An exemplary consumer device is shown in FIG. 7.

Sensors from the wearable device(s) 802 and/or the consumer device(s) 804 may communicate and transmit sensor data wirelessly via a communications network, e.g., the Internet, represented by the cloud 806 to a server 808. In some embodiment, the sensors from the wearable device(s) 802 and/or the consumer device(s) 804 may communicate and transmit sensor data through a wireless communication interface to each other via Bluetooth®, WiFi®, WiMAX®, LTE, 4G, 5G and beyond, or the like. In some embodiments, the transmitted data may be encrypted prior to transmission over the network. For example, in the case of patient data, the data collected at the sensors described herein may be encrypted to, e.g., comply with the Health Insurance Portability and Accountability Act (HIPAA), prior to transmission.

One or more servers 808 may retrieve sensor data from the cloud 806 and may analyze and process the sensor data using a data processing module. Servers 808 may then send the analyzed (and encrypted) data through cloud 806 to any suitable device such as, e.g., a smart watch, a smart phone, or a computer.

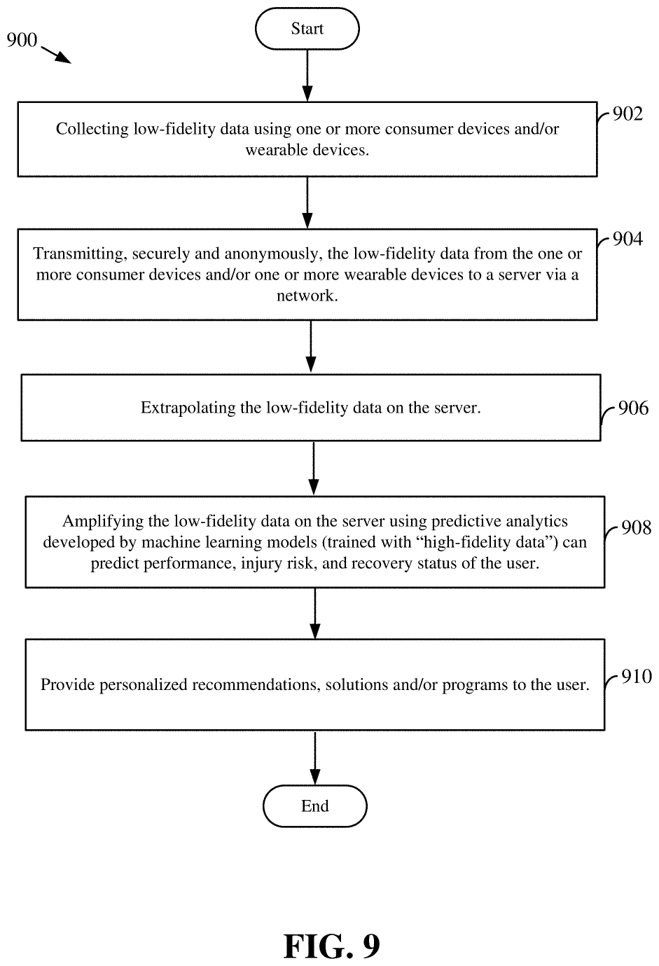

FIG. 9 is a flow chart illustrating an example of a process (e.g., a method) of providing sophisticated analysis and insights on various aspects of the body leading to more relevant and useful predictions on performance improvement and injury risk while continuously monitoring the progress, according to some aspects of the disclosure. As described below, some or all illustrated features may be omitted in a particular implementation within the scope of the present disclosure, and some illustrated features may not be required to implement all examples. In some examples, the process 900 may be carried out by any suitable apparatus or means for carrying out the functions or algorithm described below.

At 902, the process 900 may include the one or more consumer devices and/or wearable devices collecting, for example, low-fidelity. Sets of instructions have been exemplified above and their description will not be repeated for the sake of brevity. At 904, the one or more consumer devices and/or wearable devices transmits the low-fidelity data to a server via a network. According to some aspects, the low-fidelity data is transmitted securely and anonymously to the server.

At 906, the low-fidelity data is extrapolated by the server. At 908, the server amplifies the low-fidelity data using predictive analytics, developed by machine learning models that have been trained with high-fidelity data, to predict performance, injury risk, and recovery status of the user. The predicted data related to performance, injury risk, and recovery status is by way of example only and other data may be predicted.

At 910, the server provides personalized recommendations, solutions, and/or programs are provided to the user. Although data collection will happen from a singular (one) or plurality of (many) wearables, wearable devices, and/or consumer devices, the aggregated information collected will be extrapolated in the cloud and converted to actionable insights, which will be delivered in the form of reports that can be visualized on software applications running on devices for example but not limited to phones, watches, tablets, laptops, VR (virtual reality) headsets, AR (augmented reality) devices, etc.

According to some aspects, an AI Chat Bot may be used to assess all performance, injury risk, and recovery insights across multiple trials/tests/assessments/reports to help answer in real-time many questions a user may have. For example, the user may ask (1) what shoe should I wear?; (2) what terrain should I work out on?; (3) what orthotic should I wear?; (4) which prosthetic device and/or assistive device works best for me?; (5) what should I eat/drink before my workout/competition/workday?; (6) how much sleep do I need before my workout/competition/workday?; and (7) any other question relating to a user wanting to know more about their health/wellness/fitness, wherein large volumes of context and private data corpus will be further provided to train the Large Language Models (LLM) (for example like Open AI's ChatGPT) and have it answer very specific questions for the user, including but not limited to, “how did I do today vs. 3 months ago while running a 10k”, and/or “show we a chart that explains how my knee has recovered post-surgery over 3 months”, etc. in very human like readable sentences, which will redefine the way users ask questions and get answers back from computers along with purchasing and subscribing to third party and affiliate partner products and services individualized, personalized, and tailored for them.

Novel features of the present disclosure include, but are not limited to:
1) Provide sophisticated, comprehensive, and useful insights to users: The use of a software-only approach to “supercharge” the capabilities of existing consumer devices and wearables, so that they can provide sophisticated analysis and insights on various aspects of the body leading to more relevant and useful predictions on performance improvement and injury risk while continuously monitoring the progress;
2) The non-reliance of additional sensors to get high-quality data. Today, high quality data is obtained by the use of additional and sophisticated sensors, but this adds additional cost and inconvenience for the user. This invention relies on the use of the existing sensors in the consumer devices and wearables, with the need for no additional sensors, instead relying on Machine Learning to teach the existing devices on how to “upscale” existing data, by using the comprehensive database as disclosed in the '837 patent and additional sources;
3) Broad applicability and utility: The invention will potentially allow hundreds of millions of “existing” consumer devices and wearables to get very sophisticated, since there is no additional hardware or sensor upgradation to be done. The novelty of the invention using a software only method will allow these existing devices to be upgraded with down-the-wire capability and over the air (OTA) updates; and
4) Cost reduction: The use of this invention to “supercharge” existing consumer devices and wearables could potentially help deliver sophisticated outcomes at significantly lower costs, since this invention does not need expensive hardware or sensor-upgrades and can enable truly remote operation for applications like “telemedicine”, “telehealth”, “remote diagnosis” and/or “remote rehabilitation”. This could enable lower healthcare costs, overall.

Some example applications of this disclosure to deliver value to users include, but are not limited to, the following:
1) A runner, who uses a smart watch to count the steps taken and distance run, can potentially get very timely warnings of injury risk with the way his feet land on the ground. This could further lead to a shoe recommendation to fix a potential problem/concern;
2) A person who wears a wearable device at the gym while doing his weight protocol, could potentially be able to understand, based on a prescribed ‘gold-standard-protocol’, how he/she/they is/are deviating from the gold standard protocol, and what changes need to be done to ensure better results while training;
3) A person who wears a wearable device while exercising or while working out, could potentially understand the onset of fatigue along with potential injuries waiting to happen if the user continued to push through;
4) A person who wears a wearable device and works in a physically demanding environment, could potentially understand the injury risk in a specific segment or joint (symptom), and furthermore get feedback on the causes to help mitigate the symptom. For example, including but not limited to, knee pain (symptom) due to wrong footwear (cause);
5) A person who is using a wearable device or carrying a phone on the body, while performing their day-today chore, could potentially understand poor posture and get suggestions on corrective methods. For example, including but not limited to, this person may be a sedentary individual, an athlete, a weekend warrior, an industrial worker, etc.; and
6) A user is carrying a smartphone in the pant pocket and wearing a smartwatch on the wrist and has downloaded software application disclosed herein on both devices. The sensors available (i.e., factory installed) in these devices track the user's movements along with other health vitals. The data is collected by the consumer devices, synced up, and then uploaded to the cloud. At the cloud, the data is cross-referenced across one or more larger data sets to check for performance, injury risk, and recovery status. Upon completing the processing, actionable insights and recommendations are generated specific to the user. These insights and recommendations are then converted to reports (with scoring to compare a user specific to their population as per gender, age, height, weight, etc.). The reports are then delivered for visualization on the devices. The user can further ask complex questions relating to their health, wellness, and/or fitness to an AI Chat Bot and receive answers in very human like readable sentences. The user can then use these individualized, personalized and tailored answers to purchase/subscribe to products and services offered by third party health/wellness/fitness companies and affiliated partners.

While certain exemplary embodiments have been described and shown in the accompanying drawings, it is to be understood that such embodiments are merely illustrative of and not restrictive on the broad invention, and that this invention is not be limited to the specific constructions and arrangements shown and described, since various other modifications may occur to those ordinarily skilled in the art.

1. A system for collecting data related to user movements, comprising:
at least one wearable device, wherein the at least one wearable device comprises at least one sensor to collect low-fidelity data;
one or more servers for receiving the low-fidelity data via a communication network, the one or more servers comprising:
one or more databases for storing instructions for processing the low-fidelity data;
one or more processors, in communication with the one or more databases, configured to execute the instructions to perform a method, including:
collect the low-fidelity data from the at least one wearable device;
transmit the low-fidelity data from the at least one wearable device to the one or more servers;
extrapolate the low-fidelity data using predictive analysis developed by machine learning models trained with high-fidelity data;
amplify the low-fidelity data;
provide personalized recommendations to a user using the amplified low-fidelity data.

2. The system of claim 1, wherein the at least one wearable device includes a consumer device.

3. The system of claim 1, wherein the low-fidelity data is transmitted securely and anonymously to the at least one server.

4. The system of claim 1, wherein the personalized recommendations include performance, injury risk, and recovery status of the user.

5. The system of claim 1, wherein the at least one wearable device includes a wireless transmitter configured to transmit the low-fidelity data collected.

6. The system of claim 1, wherein the at least one wearable device includes a consumer device and an article of clothing, an accessory or an implant.

7. The system of claim 1, wherein the at least one sensor includes at least one of an accelerometer, a gyroscope, a magnetometer, and a heart rate sensor.

8. The system of claim 1, wherein the one or more processors is further configured to:
measure cognitive health and balance of the user;
assess the cognitive health and balance of the user; and
quantity the cognitive health and balance of the user.

9. The system of claim 1, further comprising an artificial chat bot in communication with the one or more servers to assess all performance, injury risk, and recovery insights to answer questions from the user in real-time.

10. The system of claim 9, wherein multiple trials, tests, assessments, and reports are utilized to answer the questions in real-time.

11. A system for collecting data related to user movements, comprising:
at least one wearable device, wherein the at least one wearable device comprises at least one sensor to collect low-fidelity data;
at least one consumer device, wherein the at least one consumer device comprises at least one sensor to collect low-fidelity data;
one or more servers for receiving the low-fidelity data via a communication network, the one or more servers comprising:
one or more databases for storing instructions for processing the low-fidelity data;
one or more processors, in communication with the one or more databases, configured to execute the instructions to perform a method, including:
collect the low-fidelity data from the at least one wearable device;
transmit the low-fidelity data from the at least one wearable device to the one or more servers;
extrapolate the low-fidelity data using predictive analysis developed by machine learning models trained with high-fidelity data;
amplify the low-fidelity data;
provide personalized recommendations to a user using the amplified low-fidelity data.

12. The system of claim 11, wherein the low-fidelity data collected from the at least one wearable device and the at least one consumer device is transmitted securely and anonymously to the at least one server.

13. The system of claim 11, wherein the at least one wearable device and the at least one consumer device communicate with each other directly.

14. The system of claim 11, wherein the at least one wearable device includes an article of clothing, an accessory, or an implant.

15. The system of claim 11, wherein the at least one wearable device includes a wireless transmitter configured to transmit the low-fidelity data collected.

16. The system of claim 11, wherein the low-fidelity data is transmitted securely and anonymously to the at least one server.

17. The system of claim 11, wherein the one or more processors is further configured to:
measure cognitive health and balance of the user;
assess the cognitive health and balance of the user; and
quantity the cognitive health and balance of the user.
